(19) United States
(12) Patent Application Publication
(10) Pub. No.: US 2010/0023081 A1
(43) Pub. Date: Jan. 28, 2010

(54) Comprehensive System for Detection of Coronary Syndrome, Cardiac Ischemia and Myocardial Infarction

(76) Inventors: Sarah Anne Audet, Shoreview, MN (US); James Kevin Carney, Brooklyn Park, MN (US); William J. Combs, Minnetonka, MN (US); Eduardo N. Warman, Maple Grove, MN (US); Edward Chinchoy, Golden Valley, MN (US); Thomas J. Mullen, Andover, MN (US); Brian Bruce Lee, Golden Valley, MN (US); Qingshan (Sam) Ye, Plymouth, MN (US)

Correspondence Address: MEDTRONIC, INC., 710 MEDTRONIC PARKWAY NE, MINNEAPOLIS, MN, 55432-9924, US

(21) Appl. No.: 12/181,247

(22) Filed: Jul. 28, 2008

(51) Int. Cl. A61N 1/365

(52) U.S. Cl. 607/18

(57) ABSTRACT

Heart-monitoring systems, apparatus, and methods adapted to detect CS, CI and/or MI. In one embodiment, a system comprising at least two first-tier sensors capable of measuring and converting into signals at least two aspects related to cardiac function, at least one second-tier sensor that is also a first-tier sensor, at least one signal processor capable of transmitting a first-tier and second-tier trigger signal when coronary syndrome, cardiac ischemia or myocardial infarction has been detected, at least one communication device capable of communicating, at least one control element adapted to produce a first-tier and second-tier trigger signal when at least one first-tier sensor exceeds its threshold signal level, to exclude the signal from the first-tier sensor that exceeded its threshold and lower at least one threshold of the at least one first-tier sensor is provided.

### TECHNICAL FIELD

Embodiments of the inventive subject matter relate to heart-monitoring systems, apparatus, and methods, and more particularly to heart-monitoring systems, apparatus, and methods adapted to detect acute and chronic coronary syndrome, cardiac ischemia and/or myocardial infarction.

### BACKGROUND

Numerous types of heart-related disease conditions exist, which can lead to a need for acute professional medical intervention to avoid death. As a primary example, coronary artery disease (CAD), also called ischemic heart disease, a build up of fatty deposits called plaque in the coronary arteries, which narrow the openings in these coronary arteries and cause decreased blood flow to the heart muscle. Individuals with CAD may eventually experience myocardial ischemia, an imbalance between myocardial oxygen demand and myocardial oxygen supply. Myocardial ischemia can result in irreversible cardiac cell death (e.g., cell necrosis), a result commonly referred to as a myocardial infarction. Acute myocardial infarction (AMI) is the acute phase of a myocardial infarction (MI), during which cell necrosis occurs.

An AMI is also commonly referred to as a heart attack. The resulting ischemia (restriction in blood supply) and oxygen shortage, if left untreated for a sufficient period, can cause damage and/or death (infarction) of heart muscle tissue (myocardium).

Along with CAD, other heart conditions also may result in myocardial ischemia and/or AMI. For example, myocardial ischemia may also result from pulmonary disease, hereditary heart disease, hypertensive heart disease, inflammatory heart disease, valvular heart disease, tachycardia, and hypertension, hypotension, thromboembolism, compression of a blood vessel or artery (e.g., by a tumor), foreign matter within the cardiovascular system, sickle cell disease, and/or other causes. Minimizing the time to diagnose an ST segment elevated AMI (STEMI) and to provide treatment is critical to preventing damage to the cardiac tissue and death. Diagnoses and treatment within the first hour after a STEMI event have been shown to prevent or minimize the damage to the heart caused by an MI (See JAMA, 2005, 293.797-986; Arch Intern Med/Vol. 159, 1999).

Important to the diagnosis of an MI, or ischemia, is acute coronary syndrome (ACS), which displays a whole possible set of signs and symptoms associated with a myocardial infarction or angina. They include chest pain, called angina, which may feel like a squeezing vise or crushing pressure deep in the chest behind the breastbone (sternum) and may also be felt in the shoulders, arms, back, neck or jaw. In women, angina may be experienced as abdominal pain or inconsistent chest pain that does not fit the classic model of heart-related chest pain. Other patients experience a kind of chest discomfort or pain (called angina) with their ischemia. It is believed that episodes of angina may last between two and 20 minutes. The discomfort or pain is often described as “crushing” or “heaviness.” There may be associated diaphoresis (sweating), nausea and vomiting, as well as shortness of breath.

In many cases, the sensation is “atypical,” with pain experienced in different ways or even being completely absent (which is more likely in female patients and those with diabetes). Some may report palpitations, anxiety or a sense of impending doom and a feeling of being acutely ill. According to the American Heart Association, roughly 20% of patients who have heart attacks experience angina before the attack. For obvious reasons, those experiencing chronic or acute coronary syndrome might greatly benefit from immediate medical attention and proper treatment.

Contrasting the wide array of symptoms of acute coronary syndrome is another related ischemic heart condition, silent ischemia, in which the ischemia of the heart produces no symptoms whatever. In these patients, it is often important to identify the ischemia before a major heart-related health concern, such as heart attack or sudden cardiac death, occurs. The American Heart Association estimates that between three and four million Americans have experienced silent cardiac ischemia. In fact, studies have shown that silent ischemia is the most common manifestation of coronary heart disease, accounting for about 80% of ischemic episodes. Accordingly, many of the individuals who experience silent ischemia never receive the medical attention they need.

Minor episodes of cardiac ischemia sometimes cause little long-term damage to the heart, but there may be serious side effects in some patients. Cardiac ischemia can cause abnormal heart rhythms (arrhythmias), which can lead to either syncope (fainting) or cardiac arrest (the abrupt inability of the heart to pump blood) and sudden cardiac death. Severe or lengthy episodes can result in a myocardial infarction (heart attack). The collective effects of minor episodes of cardiac ischemia can potentially lead to a weakening of the heart muscle (cardiomyopathy). Because of the potentially harmful effects of cardiac ischemia, much research has been conducted on both its symptoms and available strategies for diagnosing, treating and preventing it.

Translated literally, the term “angina pectoris” means “a choking sensation of the chest.” The pain of an angina attack can be a warning sign of a more dangerous underlying cause and should be taken seriously by patients, especially if it worsens or starts to happen more often. Angina that occurs at irregular times, without provocation (e.g., physical exertion) is known as unstable angina. This is considered a major warning sign of a heart attack, and any person experiencing unstable angina should consult a physician at the earliest possible opportunity.

It is important to note that most chest pain reported to physicians is not cardiac ischemia, but due to some other cause (e.g., muscle pain, gastrointestinal issues). However, when chest pain is reported, it is important to rule out any potential cardiac problems. Unfortunately, because chest pain is a symptom of so many conditions, many of them having nothing to do with the coronary syndrome-cardiac ischemia, myocardial infarction and other heart-related conditions are sometimes misdiagnosed, mistreated, or simply ignored by the sufferer.

Ischemia, whether silent or symptomatic, is associated with increased risk for heart attack and other serious cardiac events. In people with silent ischemia, the first indication of heart trouble is frequently a major heart attack. Thus, it is often the goal of physicians to identify as many cases of silent ischemia as possible through screening and careful attention to known risk factors for heart disease. Studies have demonstrated that silent ischemia can be more common among certain ethnic or racial groups. For example, recent reports find that Asian Americans, in comparison to white Americans, experience significantly fewer episodes of ischemic chest pain. Other symptoms, however, occur more frequently, such as shortness of breath, fatigue and palpitations. Accordingly, a quick and accurate diagnosis and treatment of cardiac ischemia, is necessary to provide proper patient care.

It is well known that AMI is the leading cause of death for both men and women around the world. Some of the more accepted risk factors are previous cardiovascular disease (such as angina, a previous heart attack or stroke), older age (especially men over 40 and women over 50), tobacco smoking, high blood levels of certain lipids (triglycerides, low-density lipoprotein or “bad cholesterol”) and low high density lipoprotein (HDL, “good cholesterol”), diabetes, high blood pressure, obesity, chronic kidney disease, heart failure, excessive alcohol consumption, the abuse of certain drugs (such as cocaine), and chronic high stress levels.

Often times, if an AMI is suspected when the patient presents to an emergency care facility, the patient will receive a number of diagnostic tests, typically in a hospital or other treatment center, such as an electrocardiogram (ECG, EKG), a chest X-ray and blood tests to detect elevations in cardiac markers (which detect heart muscle damage). The markers that are often used to detect AMI are the creatine kinase-MB (CK-MB) fraction and the troponin I (TnI) or troponin T (TnT) levels. As with other adverse heart-related conditions, immediate medical attention and proper treatment can mean the difference between life and death for a person having an AMI. Accordingly, quick and accurate diagnosis and treatment of coronary syndrome, cardiac ischemia, and myocardial infarctions are essential to minimizing damage and preventing death.

Unfortunately, sometimes these and other serious heart conditions are never diagnosed, misdiagnosed, or diagnosed too late, causing serious injury and even death to those individuals experiencing these conditions.

Myocardial ischemia may be transient, sub-lethal or persistent. Transient myocardial ischemia may have a relatively short duration (e.g., minutes), with prompt reperfusion of the coronary blood vessels. Accordingly, cell necrosis does not typically occur with transient myocardial ischemia. Sub-lethal myocardial ischemia may have a significantly longer duration (e.g., weeks or months), although it is also characterized by subsequent reperfusion and no cell necrosis. Persistent myocardial ischemia, during which no reperfusion occurs of the coronary blood vessels, may result in cell necrosis and death.

Through education, many people realize that they should promptly seek medical attention at the onset of acute coronary syndrome. However, a significant number of individuals experience non-specific symptoms or “silent” myocardial ischemia, during which they do not perceive any physical symptoms of myocardial ischemia. Accordingly, an acute coronary syndrome for such an individual may proceed to an AMI before this individual becomes inclined to seek medical attention.

### BRIEF SUMMARY

An embodiment of the inventive subject matter includes a heart-monitoring system adapted to detect coronary syndrome, cardiac ischemia and/or myocardial infarction. In one embodiment a heart-monitoring system is provided, which is capable of detecting coronary syndrome, cardiac ischemia and myocardial infarction in a human. The system comprises at least two first-tier sensors capable of measuring at least two different aspects of the human body related to cardiac function and converting the at least two different aspects of the human body related to cardiac function into one or more signals, at least one second-tier sensor that is also a first-tier sensor capable of measuring at least one aspect of the human body related to cardiac function and converting the at least one aspect of the human body related to cardiac function into one or more signals, at least one signal processor capable of receiving and analyzing the one or more signals from the at least two first-tier sensors and the one or more signals from the at least one second-tier sensor and capable of transmitting or causing to be transmitted a first-tier trigger signal when coronary syndrome, cardiac ischemia or myocardial infarction has been detected in the human and a second-tier trigger signal when coronary syndrome, cardiac ischemia or myocardial infarction has been detected in the human, at least one communication device capable of communicating any combination of the one or more signals from the at least two first-tier and one second-tier sensors, baseline cardiac function data regarding normal cardiac function of the human and threshold cardiac function data indicating coronary syndrome, cardiac ischemia or myocardial infarction in the human between the at least two first-tier sensors, the at least one second-tier sensor and the at least one signal processor, at least one control element adapted to produce a first-tier trigger signal when at least one first-tier sensor exceeds or crosses its threshold signal level, the at least one control element adapted to produce a second-tier trigger signal when at least one second-tier sensor exceeds or crosses its threshold signal level after the first-tier trigger signal is produced, the at least one control element that produces a second-tier trigger signal is adapted to exclude the signal from the first-tier sensor that exceeded or crossed its threshold, producing the first-tier trigger signal and the at least one control element adapted to lower at least one threshold of the at least one first-tier sensor that is also a second-tier sensor when the first-tier trigger signal is produced.

In one aspect of at least one embodiment of the inventive subject matter, the one or more of the at least two first-tier sensors includes one or more electrocardiogram (ECG) or one or more intracardiac electrogram (EGM) sensors.

In another aspect of at least one embodiment of the inventive subject matter, one of the at least two first-tier sensors is a patient activator with which the human may manually indicate the presence of a symptom suggesting a negative heart condition.

In yet another aspect of at least one embodiment of the inventive subject matter, the at least two first-tier sensors and the one second-tier sensor are selected from a group of sensors that include: (1) an ECG sensor; (2) a heart sound sensor; (3) a vibration sensor; (4) a pressure sensor; (5) a change in pressure with time (dp/dt) sensor; (6) a magnetic sensor; (7) a hall sensor; (8) a biomarker level sensor; (9) an oxygen level sensor; (10) a carbon dioxide level sensor; (11) a glucose sensor; (12) a pH sensor; (13) an electrolyte sensor such as Na+, K+, or Ca+; (14) a temperature sensor; (15) a heart rate sensor; (16) a heart rate variability sensor; (17) a heart wall motion sensor; (18) an impedance sensor; (19) an optical or other type of electromagnetic radiation sensor; (20) a respiration sensor; (21) a 3-axis accelerometer; (22) a neurohormone sensor; (23) an external noise sensor; (24) a patient activator; and (25) optical color reflectometry sensor.

In yet another aspect of at least one embodiment of the inventive subject matter, the electrodes can be placed on the surface of the skin, under the skin, or in or around the heart.

In yet another aspect of at least one embodiment of the inventive subject matter, the sensor or apparatus contains a microphone, accelerometer, or vibration sensor placed on the surface of the skin, under the skin, or in or around the heart.

In yet another aspect of at least one embodiment of the inventive subject matter, the one or more tier-two sensors are not tier-one sensors, each additional tier-two sensor having a threshold being the signal level at or above which indicates coronary syndrome, cardiac ischemia or myocardial infarction in the human.

In yet another aspect of at least one embodiment of the inventive subject matter, a second-tier initiation control element is adapted to activate the at least one second-tier sensors when the first-tier trigger signal is produced.

In yet another aspect of at least one embodiment of the inventive subject matter, a patient alert element is adapted to produce an alert in response to the second-tier trigger signal.

In yet another aspect of at least one embodiment of the inventive subject matter, an external system notification element is adapted to send a message to an external system in response to the second-tier trigger signal.

In yet another aspect of at least one embodiment of the inventive subject matter, a cardiac stimulus element is included in the system or apparatus.

In yet another aspect of at least one embodiment of the inventive subject matter, the system or apparatus is adapted to produce a cardiac stimulus during the measurement period of the tier-two sensor in response to the tier-one trigger signal.

In yet another aspect of at least one embodiment of the inventive subject matter, the system or apparatus is adapted to provide therapy to heart tissue of the human in response to the system detecting coronary syndrome, cardiac ischemia or myocardial infarction in the human.

In yet another aspect of at least one embodiment of the inventive subject matter, the system or apparatus additionally comprises at least one data storage unit capable of storing data from the one or more signals from the at least two first-tier and one second-tier sensors, baseline cardiac function data regarding normal cardiac function of the human and threshold cardiac function data for each of the at least two first-tier sensors, the threshold being the signal level at or above which indicates the possible presence of coronary syndrome, cardiac ischemia or myocardial infarction in the human.

In another embodiment of the inventive subject matter an apparatus is provided, comprising one or more electrocardiogram (ECG) sensors adapted to sense bioelectrical activity, to produce one or more current ECG waveforms, and to produce an ECG trigger signal when at least a portion of one of the current ECG waveforms meets, exceeds or crosses a predetermined threshold level indicating coronary syndrome, cardiac ischemia or myocardial infarction in the human, one or more heart sound sensors adapted to sense heart sounds, to produce a current heart sound waveforms, and to produce a heart sound trigger signal when at least a portion of one of the current heart sound waveforms meets, exceeds or crosses a predetermined threshold indicating coronary syndrome, cardiac ischemia or myocardial infarction in the human and a control element adapted to reduce the threshold for when at least a portion of the current ECG waveform indicates coronary syndrome, cardiac ischemia or myocardial infarction in the human when a heart sound trigger signal is produced and reduce the threshold for when at least a portion of the current heart sound waveform indicates coronary syndrome, cardiac ischemia or myocardial infarction in the human when an ECG trigger signal is produced.

In yet another aspect of at least one embodiment of the inventive subject matter, the one or more ECG sensors are adapted to produce the ECG trigger signal when a difference between an ST segment of the current ECG waveform and an ST segment of a baseline ECG waveform meets, exceeds or crosses a threshold value.

In yet another aspect of at least one embodiment of the inventive subject matter, a patient alert element is adapted to produce an alert in response or provide a heart-related therapy to the response-invoking signal.

In yet another embodiment of the inventive subject matter, a method is provided for heart monitoring, adapted to detect coronary syndrome, cardiac ischemia or myocardial infarction in the human. The method comprises sensing at least two first inputs related to cardiac function, sensing at least one second input related to cardiac function when at least one first input indicates coronary syndrome, cardiac ischemia or myocardial infarction and producing a patient alert or providing a heart-related therapy when the at least one second input also indicates coronary syndrome, cardiac ischemia or myocardial infarction.

In yet another aspect of at least one embodiment of the inventive subject matter, sensing the at least two first inputs and at least one second input comprises sensing bioelectrical activity of a heart, producing a current ECG waveform, producing an ECG trigger signal when at least a portion of the current ECG waveform indicates the coronary syndrome, cardiac ischemia or myocardial infarction, sensing heart sounds, producing a current heart sound waveform and producing a heart sound trigger signal when at least a portion of the current heart sound waveform indicates the coronary syndrome, cardiac ischemia or myocardial infarction.

In yet another aspect of at least one embodiment of the inventive subject matter, the method of heart monitoring further includes determining that the at least one first input indicates coronary syndrome, cardiac ischemia or myocardial infarction in the human when a difference between the at least one first input and the baseline information exceeds or crosses a threshold, adjusting at least one threshold used to determine whether the at least one second input indicates the coronary syndrome, cardiac ischemia or myocardial infarction, sending a message to an internal or external notification system when the at least one second input also indicates coronary syndrome, cardiac ischemia or myocardial infarction.

In yet another aspect of at least one embodiment of the inventive subject matter, the method of heart monitoring further includes initiating cardiac stimulus or therapy when the at least one second input also indicates coronary syndrome, cardiac ischemia or myocardial infarction.

In yet another aspect of at least one embodiment of the inventive subject matter, the first-tier sensors of the system, apparatus or method can also be second-tier sensors.

In another aspect of the at least one embodiment of the inventive subject matter, the sensors also analyze any inputs or signals they receive.

In yet another aspect of at least one embodiment of the inventive subject matter, when the system determines that at least one signal from the first-tier sensors has met, exceeded or crossed the threshold signal level data indicating coronary syndrome, cardiac ischemia or myocardial infarction in the human, the threshold is lowered for determining coronary syndrome, cardiac ischemia or myocardial infarction in the human for the second-tier sensors.

In yet another aspect of the inventive subject matter, the method further comprises determining that the at least two first inputs indicate coronary syndrome, cardiac ischemia or myocardial infarction in the human when a difference between the at least one first input and the baseline information exceeds or crosses a threshold, adjusting at least one threshold used to determine whether the at least one second input indicates myocardial ischemia and sending a message to an internal or external notification system when the at least one second input also indicates coronary syndrome, cardiac ischemia or myocardial infarction.

In yet another aspect of the inventive subject matter, the method includes initiating cardiac stimulus or therapy when the at least one second input also indicates coronary syndrome, cardiac ischemia or myocardial infarction.

In yet another aspect of the inventive subject matter, the method includes initiating cardiac stimulus when at least one of the at least two first-tier sensors indicates coronary syndrome, cardiac ischemia or myocardial infarction, the results of the stimulus being measured by the tier-two sensors.

In yet another aspect of the inventive subject matter, the method includes initiating cardiac stimulus when at least one of the at least two first-tier sensors indicates coronary syndrome.

In yet another aspect of the inventive subject matter, the methods and systems disclosed herein include three or more sensors and/or sensed outputs to be positive in order to generate a trigger signal and/or alarm indicating a potentially adverse event.

### BRIEF DESCRIPTION OF THE DRAWINGS

The inventive subject matter will be described in conjunction with the following drawing figures, in which:

FIG. 1 illustrates the interior anatomy of a human heart;

FIG. 2 illustrates an ECG waveform and a corresponding sound waveform for two normal cardiac cycles;

FIG. 3 illustrates a perspective view of heart-monitoring system in accordance with an example embodiment of the inventive subject matter;

FIG. 4 illustrates a functional block diagram of a coronary syndrome, cardiac ischemia or myocardial infarction detection system or apparatus, in accordance with an example embodiment;

FIG. 5 illustrates a functional block diagram of a coronary syndrome, cardiac ischemia or myocardial infarction detection system or apparatus, in accordance with an example embodiment; and

FIG. 6 illustrates a flowchart of a method for detecting coronary syndrome, cardiac ischemia or myocardial infarction, in accordance with another example embodiment.

### DETAILED DESCRIPTION

The following detailed description of the inventive subject matter is merely exemplary in nature and is not intended to limit the inventive subject matter, claims or the application and uses of the inventive subject matter. Furthermore, there is no intention to be bound by any theory presented in the preceding background or the following detailed description.

Embodiments of the inventive subject matter include systems, apparatus, and methods adapted to detect the onset or presence of coronary syndrome, cardiac ischemia or myocardial infarction. In particular, embodiments include systems, apparatus, and methods adapted to sense and analyze one or more of various physical, bioelectrical, biological, and/or chemical events or aspects that may indicate the onset or presence of coronary syndrome, cardiac ischemia or myocardial infarction. It should be appreciated that the events or aspects sensed and analyzed in the various embodiments disclosed herein include, but are not limited to, sensing and analyzing physiological signals related to coronary syndrome, cardiac ischemia or myocardial infarction in the human. In a particular embodiment, which will be described later in detail, a myocardial ischemia-detection apparatus may include an ECG sensing apparatus and a heart sound-sensing apparatus. In various embodiments, a myocardial ischemia-detection apparatus may include one or more additional sensing/analysis apparatus, including but not limited to one or more vibration sensors, pressure sensors, change in pressure with time (dp/dt) sensors, magnetic sensors, biomarker level sensors (e.g., cardiac troponin I and T, creatine phosphokinase, creatine phosphokinase myoglobin band, myoglobin, fatty acid binding protein, ischemia modified albumin, and/or lactic acid sensors), oxygen level sensors, carbon dioxide level sensors, glucose sensors, pH sensors, body temperature sensors, heart rate sensors, heart rate variability sensors, heart wall motion sensors, impedance sensors, optical sensors, respiration sensors, and/or patient activators, to name a few.

It should be appreciate that the sensor includes, among other things, one or more ECG signals, which is an electrical signal between any pair of two electrodes. It should also be appreciate that in the various multiple lead methods, apparatus and systems disclosed herein, the different leads can be or have different “vectors” because they are associated with the different orientation of the leads in the body and these different vectors can be measured separately or together.

It should also be appreciated that for purposes of the present disclosure, the ECG signal is a measurement of the electrical activity of the heart using electrodes placed on the skin (e.g., using patches), under the skin (e.g., subcutaneous), or in or around the heart (e.g., intracardiac electrograms). Accordingly, for all the embodiments disclosed herein, any type of ECG or EGM signal could be measured.

A coronary syndrome, cardiac ischemia or myocardial infarction-detection apparatus may be a stand-alone apparatus, or it may be incorporated into a host system, such as an implantable pulse generator (IPG, also known as a “pacemaker”), an implantable cardiac resynchronization therapy device (CRT), an implantable cardioverter defibrillator (ICD), an implantable cardiac diagnostic and monitoring device, a unit that combines two or more of the aforementioned devices, or another device. In addition or alternatively, a coronary syndrome, cardiac ischemia or myocardial infarction detection apparatus and/or a host system may communicate with one or more remote devices, as will be described later in more detail. In order to describe embodiments of the inventive subject matter clearly, a description of a human heart and its functioning is included below.

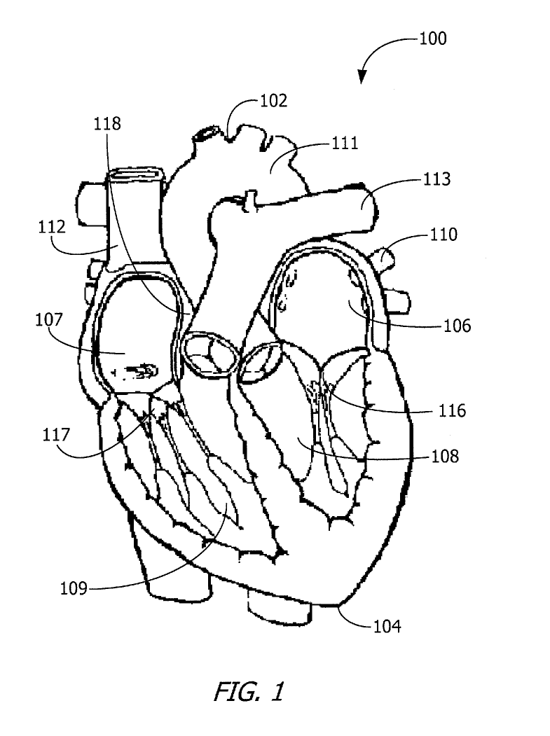

FIG. 1 illustrates the interior anatomy of a human heart 100. The top of the heart 100 may be referred to as the base 102, and the bottom of the heart may be referred to as the apex 104. The heart 100 includes four chambers: a left atrium 106; a right atrium 107; a left ventricle 108; and a right ventricle 109. During a cardiac cycle, the heart chambers 106-109 contract and relax in response to electrical currents periodically conveyed by a biological conduction system (not illustrated).

During a portion of the cardiac cycle referred to as diastole, the left atrium 106 relaxes and fills with blood from the lungs via the upper pulmonary vein 110 (i.e., pulmonary veins from the left and right lungs). The right atrium 107 also relaxes during diastole, and fills with blood from the body via the superior vena cava 112 and the inferior vena cava (not shown in FIG. 1). The blood within the left atrium 106 enters the left ventricle 108 through the mitral valve 116. The left ventricle 108 subsequently pumps the blood through the aortic valve 118 (hidden in FIG. 1) and into the body via the aorta 111 during a phase of the cardiac cycle referred to as systole.

During a portion of the cardiac cycle referred to as diastole, the blood within the right atrium 107 enters the right ventricle 109 through the tricuspid valve 117. The right ventricle 109 subsequently pumps the blood through the pulmonary valve 119 and into the lungs via the pulmonary artery 113 during systole.

The set of four heart valves functions to regulate blood flow through the chambers 106-109 of the heart 100, by opening and closing at various times. These valves include the atrioventricular (AV) valves 116, 117 and the semilunar (SL) valves 118, 119. The AV valves include the mitral valve 116 and the tricuspid valve 117. The AV valves 116, 117 open during diastole to allow the ventricles to fill with blood. The SL valves 118, 119, which are set between the arteries 106, 107 and the ventricles 108, 109, include the aortic valve 118 and the pulmonary valve 119. The SL valves 118, 119 open during systole to allow blood to be ejected from the heart 100.

During a cardiac cycle (i.e., a heartbeat), a sequence of events occurs within the heart 100, as described above. As mentioned previously, each cardiac cycle includes a diastole portion and a systole portion. More specifically, each cardiac cycle includes three major stages: atrial systole; ventricular systole; and complete cardiac diastole. These three stages may be monitored by an ECG, which senses the pattern of electrical impulses generated in the heart during the stages of the cardiac cycle. A series of “heart sounds” (also referred to as “heart tones”), which occur synchronously with corresponding portions of an ECG, also may be sensed and represented by a sound waveform.

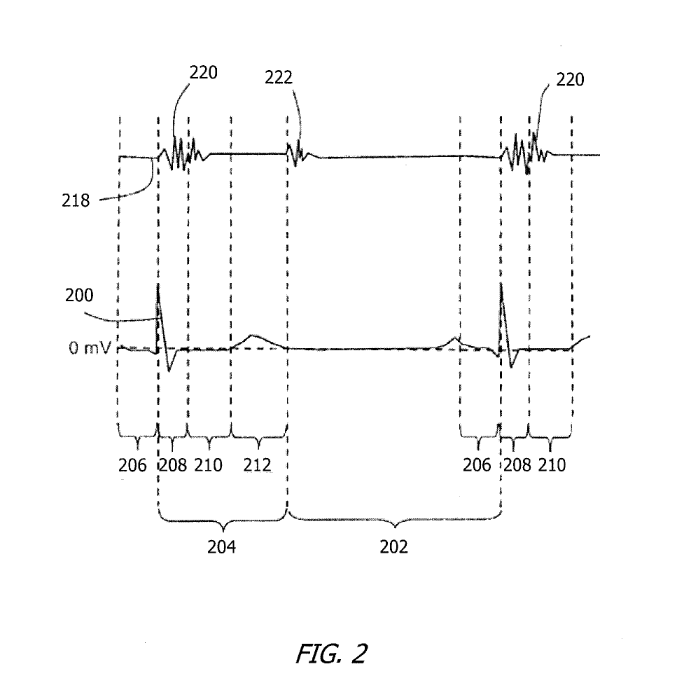

FIG. 2 illustrates an ECG waveform 200 and a corresponding sound waveform 218 for two normal cardiac cycles. A cardiac cycle includes TR segment 202 and a QT segment 204. The TR segment 202 includes a P wave 206. The P wave 206 indicates the movement of electrical activity through the upper heart chambers. Atrial systole occurs at the onset of the P wave 206, and indicates the contraction of the myocardium (i.e., heart muscle tissue) of the left and right atria. Atrial systole includes an electrical systole (i.e., the electrical activity that stimulates contraction of the myocardium of the heart chambers) followed by a mechanical systole (i.e., the mechanical contraction of the heart chambers).

Complete cardiac diastole occurs during the TR segment 202, and indicates the relaxation of the heart muscle after contraction in preparation for refilling with circulating blood. Complete cardiac diastole includes a ventricular diastole period when the ventricles are relaxing, and an atrial diastole period when the atria are relaxing. During the ventricular diastole period, the blood pressure in the left and right ventricles drops from the peak that it reached in systole. When the pressure in the left ventricle drops to a pressure below the pressure in the left atrium, the mitral valve opens, and the left ventricle fills with the blood that was accumulating in the left atrium. Likewise, when the pressure in the right ventricle drops below the pressure in the right atrium, the tricuspid valve opens, and the right ventricle fills with the blood that was accumulating in the right atrium.

The QT segment 204 of a cardiac cycle includes a QRS complex 208, ST segment 210, and T wave 212. The ST segment 210 normally appears as a straight, level line between the QRS complex 208 and the T wave 212. The T wave 210 corresponds to the period when the lower heart chambers are relaxing electrically and preparing for their next muscle contraction.

Ventricular systole occurs during QT segment 204, and indicates the contraction of the myocardium of the left and right ventricles. Similar to atrial systole, ventricular systole also includes an electrical systole followed by a mechanical systole. In an ECG 200, electrical systole of the ventricles begins at the beginning of the QRS complex 208 (i.e., a measurement of the movement of electrical impulses through the lower heart chambers).

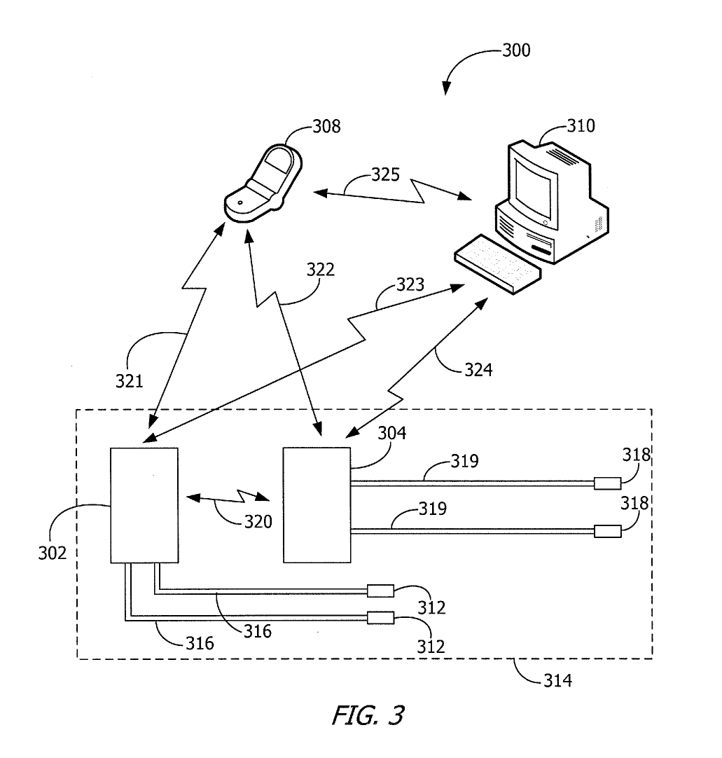

During a cardiac cycle, many distinct heart sounds may be audible. Two of these heart sounds, represented along sound wave 218, are referred to as the first heart sound 220 (S1) and the second heart sound 222 (S2). The third heart sound (S3) and the fourth heart sound (S4) will be discussed later in conjunction with FIG. 3. As used herein, an “ausculation area” refers to an area of the heart or the body where a sensor may be placed to sense the heart sounds.

The first heart sound 220 is associated with the closing of the mitral and tricuspid valves (i.e., the AV valves) at the beginning of ventricular systole. The first heart sound 220 is the first part of the “lub-dup” sound of a heartbeat. The second heart sound 222 is associated with the closing of the aortic and pulmonic valves (i.e., the SL valves) at the end of ventricular systole. The second heart sound 222 is the second part of the “lub-dup” sound of a heartbeat. At the end of ventricular systole, as the left ventricle relaxes, its pressure falls below the pressure in the aorta, and the aortic valve closes. Similarly, as the pressure in the right ventricle falls below the pressure in the pulmonary artery, the pulmonic valve closes. In a normal sequence, the aortic valve closes a few milliseconds before the pulmonic valve.

FIG. 3 illustrates a perspective view of heart-monitoring system 300, in accordance with an example embodiment of the inventive subject matter. System 300 may include at least one host system 302, sensing apparatus 304, patient communication device 308 (e.g., a radio, wristwatch, telephone, pager, patient activator), and computing device 310 (e.g., a computer), in an embodiment. In another embodiment, host system 302 and sensing apparatus 304 may be included within a single device. In other embodiments, some or all of host device 302, patient communication device 308, and/or computing device 310 may be excluded from the heart-monitoring system 300.

In an embodiment, host system 302 and/or sensing apparatus 304 may be implanted within and/or positioned externally on a patient 314. Host system 302 may be a device adapted to provide stimuli to the heart tissue, and may include, for example but not by way of limitation, implantable pulse generator (IPG), an implantable cardiac resynchronization therapy device (CRT), implantable cardioverter defibrillator (ICD), an implantable cardiac diagnostic and monitoring device, a unit that combines two or more of the aforementioned devices, or another device. Accordingly, host system 302 may include one or more stimulus elements (e.g., electrical and/or mechanical stimulus elements) 312 adapted to be implanted within a patient 314. The stimulus elements 312 may be coupled to host system 302 via leads 316, or they may have leadless (e.g., direct, wireless radio frequency, and/or ultrasound) connections to host system 302 and/or to each other. Leads 316 may include transvenous leads and/or subcutaneous tethers, in various embodiments.

Sensing apparatus 304 may include an apparatus adapted to sense one or more vibratory, mechanical, electrical, magnetic, chemical, radiant, thermal or patient-initiated activity. As used herein, the terms “sensor” and “sensing apparatus” may be used interchangeably. In an embodiment, sensing apparatus 304 may include one or more sensing elements 318 adapted to be implanted within or positioned externally to patient 314. The sensing elements 318 may be coupled to sensing apparatus 304 via leads 319, or they may have leadless (e.g., direct, wireless radio frequency, and/or ultrasound) connections to sensing apparatus 304 and/or to each other. Leads 319 may include transvenous leads and/or subcutaneous tethers, in various embodiments. Leads 316 and 319 may be the same or different leads, in various embodiments. A sensing element 318 may include, for example but not by way of limitation, a microphone, accelerometer, resonator, voltage or current detector, hall sensor, blood collection device, oxygen sensor, and/or temperature sensor. In an embodiment, a sensing element 318 also or alternatively may include a patient activator, which enables a patient to manually indicate the presence of a symptom. In an embodiment, a patient activator may have inputs adapted to enable the patient to indicate the type of symptom, as well. For example, a first input may indicate acute pectoral angina (i.e., chest pain), a second input may indicate shortness of breath, and a third input may indicate fatigue, for example. In an embodiment, different inputs may cause a different response in system 300.

Sensing apparatus 304 may further include apparatus adapted to produce information representing the sensed activity over time, in an embodiment. For example, but not by way of limitation, sensor-produced information may include a sound waveform (e.g., sound waveforms 218, 318, FIGS. 2 and 3), a vibration waveform, an ECG waveform (e.g., ECG waveforms 200, 300, FIGS. 2 and 3), a magnetic magnitude waveform, a biomarker level waveform, an oxygen level waveform, a body temperature waveform, and/or patient activator input signals (e.g., symptomatic or asymptomatic, type of symptom, etc.), or other sensor-produced information. Sensing apparatus 304 may further include apparatus adapted to analyze sensed activity, and to produce an output signal based on the analysis. Output signals may include, for example, analog or digital signals indicating values of the sensed activity, and or signals indicating that a sensed activity has reached or exceeded or crossed a threshold (or fallen below a threshold), for example.

In an embodiment, when host system 302 and sensing apparatus 304 are discrete devices, they may communicate with each other over wireless intrabody link 320. In addition, in various embodiments, either or both host system 302 and/or monitor device 304 may communicate with patient communication device 308 and external system communication device 310 over wireless extrabody links 321, 322, 323, 324. Additionally, patient communication device 308 and computing device 310 may communicate with each other over wireless device-to-device link 325. A variety of different wireless communication technologies may be used to support communication over links 320-325.

During operation, system 300 may function to sense various activity, and to produce signals representing the sensed activity, via sensing apparatus 304 and/or host system 302. The sensed activity signals may be communicated with other system elements. Host system 302 and/or the various system elements may (or may not) take action based on the sensed activity signals. An example of system 300 operation is given below. The example is not meant to limit the scope of the inventive subject matter.

In one operational scenario, sensing apparatus 304 may be adapted to sense one or more cardiac activities. For example, sensing apparatus 304 may include one or more voltage sensors, which may be positioned proximate to one or more areas of the heart in order to sense bioelectrical stimulation of the heart. Also, sensing apparatus 304 may include one or more mechanical sensors (e.g., microphones, accelerometers, pressure sensors, heart wall motion sensors, or resonators), which may also be positioned proximate to one or more areas of the heart in order to sense mechanical cardiac activity (e.g., heart sounds and/or vibration of the heart walls). Sensing apparatus 304 may include one or more additional sensors in various embodiments to sense other activities. Sensing apparatus 304 may convert the sensed bioelectrical stimuli, mechanical cardiac activity, and other sensed stimuli, if any, into signals that may be further analyzed by sensing apparatus 304, host system 302 or another system component. When host system 302 or another system component is adapted to analyze the signals, sensing apparatus 304 may transmit the signals over one or more of intrabody link 320 and/or extrabody links 321-322.

Signal analysis, which may be performed by sensing apparatus 304, host system 302, and/or another system component, may include analyzing the signals to determine whether the likelihood exists that myocardial ischemia is occurring. In addition, signal analysis may include considering other factors, such as whether a patient has indicated (e.g., via patient communication device 308) that the patient is experiencing angina (e.g., acute angina pectoris).

In an embodiment, when a likelihood exists that myocardial ischemia is occurring, one or more of the system components may take further action. For example, host system 302 may produce a stimulus to the heart tissue (e.g., defibrillation stimulus, pacing stimulus, pacing rate adjustment, pacing characteristic adjustment, and/or pulse adjustment stimulus). In addition or alternatively, patient communication device 308 and/or computing device 310 may produce an audible, visual or mechanical alert, and/or may provide instructions to the patient via a display or speaker. In other embodiments, heart rate, pulse pressure, and/or changes in pressure with time may be used to normalize baseline information and/or to adjust thresholds.

Patient communication device 308 and/or computing device 310 additionally or alternatively may connect over an external system to convey information regarding the patient to a remote individual or system. For example, patient communication device 308 and/or computing device 310 may connect over a cellular, radio, telephone, or computer network with a doctor's office, emergency response system (e.g., a 911 system), hospital, caretaker or other entity to indicate the likelihood of the myocardial ischemia. In addition, data representing the sensed stimuli may be conveyed over the external system. The remote individual or system may be able to communicate with the patient via patient-communication device 308 and/or computing device 310.

In an embodiment, likelihood is determined that coronary syndrome, cardiac ischemia or myocardial infarction is occurring by a coronary syndrome, cardiac ischemia or myocardial infarction-detection apparatus. A coronary syndrome, cardiac ischemia or myocardial infarction-detection apparatus may be included within sensing apparatus 304 and/or host system 302, in various embodiments. In other embodiments, some portions of a coronary syndrome, cardiac ischemia or myocardial infarction detection-apparatus may be included within another system component (e.g., patient communication device 308 and/or computing device 310), or portions of a coronary syndrome, cardiac ischemia or myocardial infarction-detection apparatus may be distributed across multiple system components.

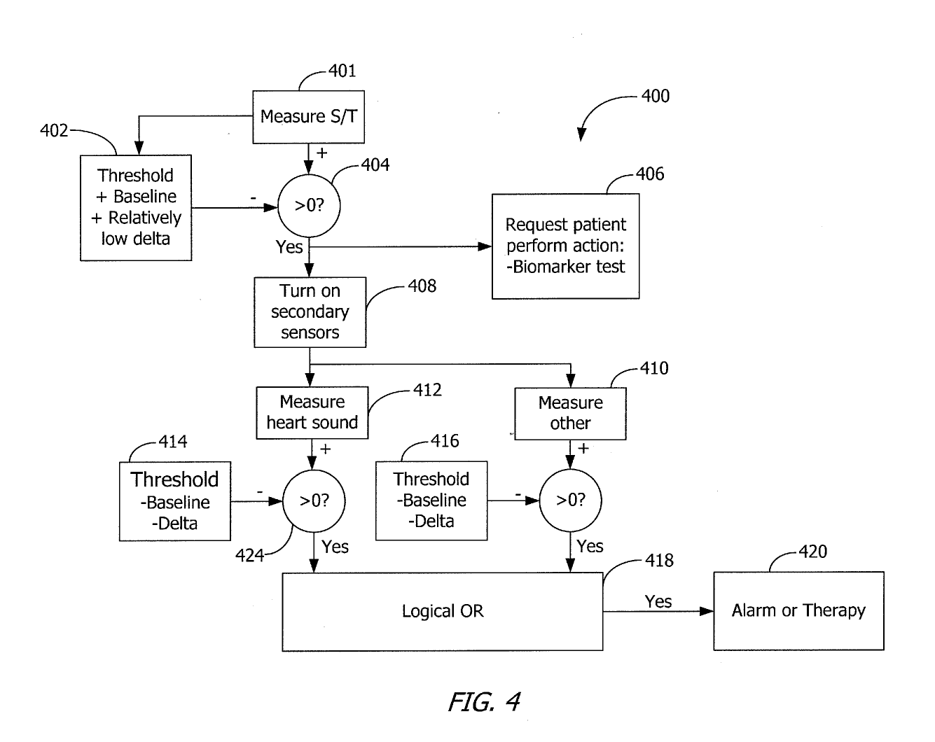

FIG. 4 illustrates a functional block diagram of a coronary syndrome, cardiac ischemia or myocardial infarction detection-apparatus, system or method 400, in accordance with an example embodiment. In various embodiments, the elements of apparatus 400 may be located in a single device package (e.g., 401-422, FIG. 4), or may be distributed among multiple device packages or systems. Additionally, interconnections between the various elements of apparatus 400 may be direct, wired or wireless, in various embodiments.

Apparatus 400 may include at least one first-tier sensor 401, 406, at least one first-tier analyzer 402, and a first-tier triggering element 404, in an embodiment. In addition, in an embodiment, apparatus 400 may include a second-tier initiation control element 408, at least one second-tier sensor 410, 412, at least one second-tier analyzer 414, a second-tier triggering element 424, a patient alert or therapy-providing element 420. It should be appreciated that apparatus 400 also includes memory, which may include one or more memory elements (e.g., random access memory (RAM), read-only memory (ROM), or other memory element). Memory is adapted to store baseline information and/or threshold values, in any embodiment. When referred to together, a sensor (e.g., sensor 401) and an analyzer 402 (e.g., analyzer) may be termed a “sensor.”

Each first-tier sensor 401, 406 is adapted to sense an input related to cardiac function, and to produce a first-tier trigger signal when the input indicates coronary syndrome, cardiac ischemia or myocardial infarction.

ECG sensor 401 may include, for example, a set of leads (e.g., 12 leads) that provide connections between a package (e.g., analyzer 402 or trigger element 404, FIG. 4) and sensors (e.g., electrodes) placed at various locations in proximity to a heart. In another embodiment, ECG sensor 401 may include leadless sensors, which are directly coupled to a package. In an embodiment, ECG sensor 401 and ECG analyzer are continuously active, meaning that ECG sensor 401 continuously senses bioelectrical activity, and ECG analyzer continuously produces a corresponding ECG waveform. In an alternate embodiment, ECG sensor 401 and/or ECG analyzer may be activated periodically or occasionally as a result of some trigger condition.

In an embodiment, ECG analyzer receives baseline information and/or one or more threshold values from memory. Baseline information may include, for example, baseline ECG information, which includes data representing ECG waveforms for one or more previous cardiac cycles. The baseline ECG information may include several sets of baseline information, including for example, baseline information representing ECG waveforms previously produced during periods of rest and/or activity, at different points in a respiration cycle, at various heart rates, and/or at different body temperatures. Desirably, the baseline ECG information represents ECG waveforms produced while the patient was not encountering coronary syndrome, cardiac ischemia or myocardial infarction.

ECG analyzer produces a current ECG waveform from bioelectrical activity sensed by ECG sensor 401, and compares the current ECG waveform to the baseline ECG information retrieved from memory. In an embodiment, ECG analyzer may select a particular set of baseline ECG information based on the patient's level of activity, the point in the respiration cycle, heart rate, among other things. In another embodiment, a single set of baseline ECG information may be stored in memory, and such a selection may not be performed. Comparison of the current ECG waveform and the baseline ECG information may include performing a correlation algorithm, in an embodiment, which may compensate for timing delays and pacing differences between the current ECG waveform and the baseline ECG information. ECG analyzer may produce a comparison result, in an embodiment, which indicates differences between the current ECG waveform and the baseline ECG information.

ECG analyzer may additionally determine whether the comparison result has reached, exceeded or crossed a threshold, referred to herein as an “ECG result threshold,” which may indicate that coronary syndrome, cardiac ischemia or myocardial infarction is occurring. An ECG result threshold may indicate a different value between corresponding portions of the current ECG waveform and the baseline ECG information. In an embodiment, for example, a particular surface ECG result threshold may be a difference of 0.1 mV or more between a baseline ST segment (e.g., ST segment 210, FIG. 2) and a current ST segment. In another embodiment, a surface ECG result threshold may be a difference of −0.1 mV or more between a baseline T wave (e.g., T wave 212, FIG. 2) and a current T wave. In an embodiment, when an ECG result threshold is reached, exceeded or crossed, then ECG analyzer produces an ECG trigger signal. Processing of the ECG trigger signal is well known in the art.

In the embodiments discussed above, an ECG trigger signal is produced based on whether a comparison of a current ECG waveform and baseline ECG information yields a comparison result that exceeds or crosses one or more ECG result thresholds. In an alternate embodiment, a current ECG waveform may be compared directly to one or more ECG result thresholds. Accordingly, one or more portions of a current ECG waveform may be analyzed to determine, for example, whether voltages during those portions meet, exceed, or are less (e.g. crosses) than one or more ECG result thresholds. For example, a determination may be made whether the voltage for a current ST segment is equal to or greater than an ST segment threshold (e.g., 0.1 mV or some other threshold). As another example, a determination may be made whether a T wave is below 0 mV (i.e., the T wave is inverted). In an embodiment, when an ECG result threshold is reached or exceeded, then ECG analyzer produces an ECG trigger signal.

It should be appreciated that in any of the embodiments disclosed herein, exceeding or crossing the threshold includes both moving threshold from a low to high level or value and from a high to low level or value.

In an embodiment, an ECG sensor 401 and an ECG analyzer are the only first-tier sensor and first-tier analyzer included in system. In other embodiments, one or more additional first-tier sensors 401, 406 and first-tier analyzers may be included in system 400. For example, but not by way of limitation, the one or more additional first-tier sensors may include any combination of one or more sensors and/or analyzers adapted to sense and analyze one or more vibratory, mechanical, electrical, magnetic, chemical, radiant, thermal or patient-initiated activity (e.g., a patient indicating the presence of symptoms through a user interface of a patient activator).

In a particular embodiment, an additional first-tier sensor includes one or more vibration sensors (e.g., a heart sound sensors), and a corresponding additional first-tier analyzer includes vibration processing electronics (e.g., heart sound analysis electronics).

Heart sound sensor may include, for example, a set of leads that provide connections between a package (e.g., host system 402 or sensing apparatus 404, FIG. 4) and sensors placed at various locations in proximity to a heart. Sensors may include, for example but not by way of limitation, any combination of one or more microphones, accelerometers, and/or resonators. In another embodiment, heart sound sensor may include leadless sensors, which are directly coupled to a package. In an embodiment, heart sound sensor and heart sound analyzer are continuously active, meaning that heart sound sensor continuously senses vibratory cardiac activity, and heart sound analyzer continuously produces a corresponding sound wave. In an alternate embodiment, heart sound sensor and/or heart sound analyzer may be activated periodically or occasionally. For example, in an embodiment, heart sound sensor and/or heart sound analyzer may be activated during certain portions of a cardiac cycle, such as during those portions when a first heart sound, a second heart sound, a third heart sound, and/or a fourth heart sound may occur. Occasional activation of heart sound sensor and/or heart sound analyzer may help to conserve power.

In an embodiment, heart sound analyzer receives baseline information and/or one or more threshold values from memory. Baseline information may include, for example, baseline heart sound information, which includes data representing heart sound waveforms for one or more previous cardiac cycles. The baseline heart sound information may include several sets of baseline information, including for example, baseline information representing heart sound waveforms previously produced during periods of rest and/or activity, at different points in a respiration cycle, at various heart rates, at different body temperatures. Desirably, the baseline heart sound information represents heart sound waveforms produced while the patient was not encountering coronary syndrome, cardiac ischemia or myocardial infarction.

Heart sound analyzer produces a current heart sound waveform from vibratory cardiac activity sensed by heart sound sensor, and compares the current heart sound signal to the baseline heart sound information retrieved from memory. In an embodiment, heart sound analyzer may select a particular set of baseline heart sound information based on the patient's level of activity, the point in the respiration cycle, heart rate, and/or posture, among other things. In another embodiment, a single set of baseline heart sound information may be stored in memory, and such a selection may not be performed. Comparison of the current heart sound signal and the baseline heart sound information may include performing a correlation algorithm, in an embodiment, which may compensate for timing delays and pacing differences between the current heart sound signal and the baseline heart sound information. In addition, heart sound analyzer may receive current ECG waveforms and/or trigger information that enables heart sound analyzer to determine where, within a cardiac cycle, various sensed heart sounds are occurring. In an embodiment, a first heart sound may be used as a reference, as it generally has the greatest amplitude of all of the heart sounds. Heart sound analyzer may produce a comparison result, in an embodiment, which indicates differences between the current heart sound signal and the baseline heart sound information. A comparison result may include difference information for one or more heart sounds (e.g., S1, S2, S3, and/or S4). In an embodiment, difference information for a fourth heart sound (S4) is particularly represented as the new appearance and/or change in amplitude of a fourth heart sound, which may indicate current coronary syndrome, cardiac ischemia or myocardial infarction.

In other embodiments, one or more additional first-tier sensors may be included in the system, such as a vibration sensor, a pressure sensor, a change in pressure with time (dp/dt) sensor, a magnetic sensor, a hall sensor, a blood collection device and biomarker level sensor, an oxygen level sensor, carbon dioxide level sensor, a glucose sensor, a pH sensor, a temperature sensor, a heart rate sensor, a heart rate variability sensor, a heart wall motion sensor, an impedance sensor, an optical sensor, a respiration sensor, 3-axis accelerometer, external noise sensor and/or a patient activator. In addition, one or more corresponding additional first-tier analyzers may be included in system 500. The one or more additional first-tier analyzers may analyze sensor signals by comparing the sensor signals to corresponding baseline information and/or to corresponding threshold values. When a sensor signal meets or exceeds a threshold value, an additional first-tier sensor may produce a first-tier trigger signal.

In one or more embodiments, the system, apparatus or method lowers the threshold (e.g., 526, 616) for determining coronary syndrome, cardiac ischemia or myocardial infarction after one or more sensors have detected coronary syndrome, cardiac ischemia or myocardial infarction.

Second-tier initiation control element (also referred to herein as a “control element”) is adapted to activate inactivated ones of the at least one second-tier sensor when at least one first-tier trigger signal (e.g., one or more of first-tier trigger signals) is produced, and/or when a second-tier initiation signal is produced. In an embodiment, second-tier initiation control element receives the second-tier initiation signal. In response to the second-tier initiation signal, second-tier initiation control element may take any one or more of several possible actions. In an embodiment, for example, second-tier initiation control element may produce an alert signal indicating that coronary syndrome, cardiac ischemia or myocardial infarction may have been detected.

Patient alert element is adapted to receive alert signal and to provide an alert to the patient. For example, but not by way of limitation, patient alert element may produce an audible or vibratory alert. In addition or alternatively, patient alert block may produce an audible or displayed message, indicating to the patient that they should take some action. Such actions may include, for example but not by way of limitation, instructions to reduce activity, take a blood sample and perform a biomarker test, and/or seek medical attention. In an embodiment, one or more mechanical sensors (e.g., microphones, accelerometers, resonators, pressure sensors, heart wall motions sensors, or other mechanical sensors, not illustrated) that may be placed in one or more positions that are relatively insensitive to heart sounds, but that are likely to sense noise due to exterior mechanical interactions. Inputs from such sensors may be used to eliminate exterior mechanical sounds, and to increase the SNR for heart sound sensing/analysis.

In an embodiment, second-tier initiation control element also may activate one or more second-tier sensors and second-tier analyzers. Each second-tier sensor is adapted to sense an input related to cardiac function, and to produce a second-tier trigger signal when the input indicates coronary syndrome, cardiac ischemia or myocardial infarction. Second-tier sensors and second-tier analyzers may include, for example but not by way of limitation, any combination of one or more sensors/analyzers adapted to sense and analyze one or more vibratory, mechanical, electrical, magnetic, chemical, radiant, thermal or patient-initiated activity. In an embodiment, some or all of second-tier sensors and second-tier analyzers may be the same as first-tier sensors and first-tier analyzers, in which cases the hardware and circuitry may not be duplicated, and activation may not be performed.

In an embodiment, second-tier initiation control element may adjust threshold values stored in memory for those analyzers that are present in the first-tier and/or the second-tier. For example, second-tier initiation control element may adjust threshold values so that the second-tier analyzers are more sensitive (e.g., reduce the threshold values). In an alternate embodiment, second-tier analyzers may use different threshold values from those used previously. Thresholds may also be adjusted at other times and/or in other ways (e.g., in response to historical data and/or external inputs).

Similar to the functioning of first-tier analyzers, second-tier analyzers may receive and process signals from second-tier sensors, and may compare the processed signals to baseline information retrieved from memory. In addition or alternatively, second-tier analyzers may determine whether comparison results, signals or waveforms based on the second-tier sensor outputs meet or exceed corresponding threshold values (or adjusted threshold values). When a comparison result, signal or waveform does meet or exceed a corresponding threshold value (or adjusted threshold value), a second-tier analyzer may produce a second-tier trigger signal.

Second-tier triggering element receives the one or more second-tier trigger signals from the one or more second-tier analyzers. In an embodiment, second-tier triggering element includes logical OR circuitry, which produces one or more response-invoking signals when any one or more of second-tier trigger signals indicate a trigger condition. In another embodiment, second-tier triggering element may include logical AND circuitry, which produces one or more response-invoking signals when more than one and/or all of second-tier trigger signals indicate trigger conditions. In still another embodiment, in which only one second-tier sensor and second-tier analyzer is included within the system, a second-tier triggering element may be excluded.

Patient alert element may produce an audible or vibratory alert or an audible or displayed message, indicating to the patient that they should take some action. The patient alert may be different from the previous alert, if one was previously produced. For example, the patient alert may indicate that more urgent action should be performed, such as an alert to contact emergency medical personnel immediately.

In an embodiment, the therapy provided may produce a stimulus to the heart tissue (e.g., defibrillation stimulus, pacing stimulus, pacing rate adjustment, pacing characteristic adjustment, and/or pulse adjustment stimulus) or provide a medication or other useful treatment.

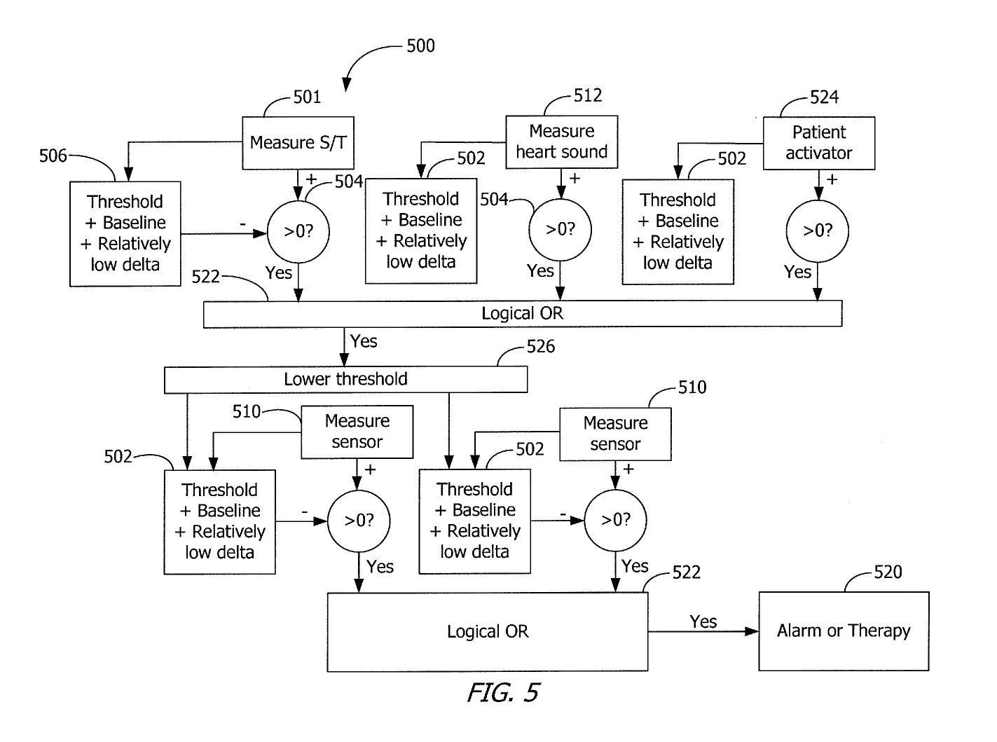

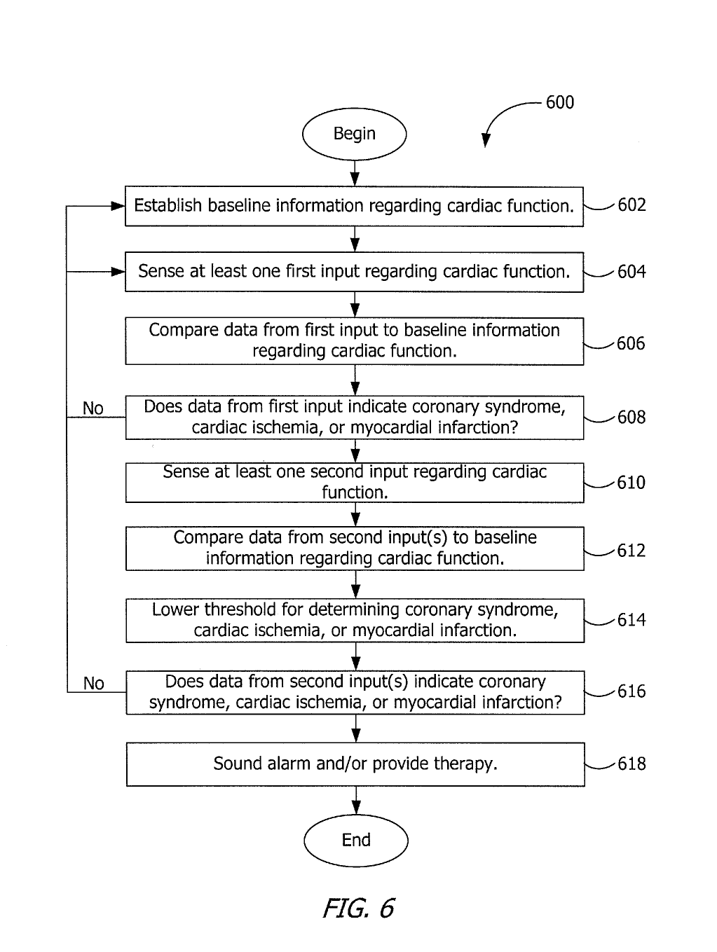

The functional block diagram of FIGS. 5 and 6 may be implemented in hardware in any of a number of ways. In particular, some or all of the various elements may be implemented using one or more processors, memory elements, logical processing blocks, and other hardware. Additionally, the various elements may be implemented in software, hardware, or both. Further, some or all of the various elements may be implemented on the same hardware platform, on different hardware platforms, using the same processor/hardware, and/or using different processors/hardware.

As discussed previously, various embodiments may include various numbers of first-tier sensors/analyzers and second-tier sensors/analyzers. In a particular embodiment, only an ECG sensor and an ECG analyzer are included as a first-tier sensor, and at least one different type of sensor is included in the second-tier sensors/analyzers. In particular, at least a heart sound sensor is included as a second-tier analyzer. Such a system may have relatively high specificity, meaning that the system may be adapted to produce an alarm, external system notification, and/or cardiac stimulus at relatively low first-tier and second-tier thresholds than other embodiments, as both thresholds are crossed in order to generate a patient alert. Method embodiments for such systems are discussed below, in conjunction with FIG. 6.

FIG. 6 illustrates a flowchart of a method for detecting coronary syndrome, cardiac ischemia or myocardial infarction, in accordance with an example embodiment. The method begins, in an embodiment, by establishing baseline information for the patient, in block 602. Establishing baseline information may include, for example, collecting and storing data representing ECG waveforms and heart sounds, among other things, for one or more cardiac cycles. As discussed previously, the baseline information may include several sets of baseline information, including for example, baseline information representing signals or waveforms produced during periods of rest and/or activity, at different points in a respiration cycle, at various heart rates, at different body temperatures. Desirably, the baseline information represents signals or waveforms produced while the patient was not encountering coronary syndrome, cardiac ischemia or myocardial infarction. Establishing baseline information may be performed one time, periodically, or continuously (as long as coronary syndrome, cardiac ischemia or myocardial infarction is not occurring), in various embodiments.

Unless already activated, a first-tier sensor (e.g., first-tier sensor 401, 405, 501, 512, 524) may be activated to sense at least one input regarding coronary function 604. Then the data from the at least one first input is compared to baseline data for the person, which could be different for each person 606. If the data from the at least one first input indicates coronary syndrome, cardiac ischemia or myocardial infarction then the at least one second input regarding cardiac function is activated and the process is continued to the next step. If the data from the at least one first input does not indicate coronary syndrome, cardiac ischemia or myocardial infarction than either the method established baseline again 602 or senses at least one first input regarding cardiac function 604. Assuming the data from the at least one first input indicates coronary syndrome, cardiac ischemia or myocardial infarction that the second sensors sense input regarding cardiac function 610 and compare that data to baseline info regarding cardiac function 612 and the threshold for determining coronary syndrome, cardiac ischemia or myocardial infarction is lowered 614. If this data also suggests that coronary syndrome, cardiac ischemia or myocardial infarction has been detected, that an alarm is sounded or therapy is provided as discussed herein 618. If not, the method repeats 602 or 604.

It should be appreciated that the triggering discussed in the various embodiments disclosed herein can be used for turning on, turning off, or modifying therapies or treatments to treat coronary syndrome, cardiac ischemia or myocardial infarction.

It should also be appreciated that in the various embodiments disclosed herein, the first-tier sensor can also be a second-tier sensor after at least one of the first-tier sensors has been activated. Accordingly, lowering the threshold for the sensor can apply to the first-tier sensor or the second-tier sensor.

It should also be appreciated that an irregular or ischemic area would change the electrical conduction pattern of the signals (e.g., paced pulse) disclosed herein. Accordingly, this change could be picked up by one or more ECG or other type of electrode. It should be appreciated that If there is more than one pair, each would be measuring a different vector of the ECG or other type of electrode spread around the body, each one looking at a different vector of the electrical signal.

It should also be appreciated that at least some of the systems, methods, and apparatus disclosed herein could be implanted or external.

In yet another embodiment, the systems, apparatus and methods disclosed herein can be used to pace the heart using methods well known in the art in response to the at least one first-tier trigger and measuring the results of the heart pace to help determine the condition of the patient and adjust the systems, apparatus and methods disclosed herein accordingly.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the inventive subject matter. Rather, the foregoing detailed description will provide those skilled in the art with a convenient road map for implementing an embodiment of the inventive subject matter, it being understood that various changes may be made in the function and arrangement of elements described without departing from the scope of the inventive subject matter as set forth in the appended claims and their legal equivalents.

What is claimed is:

1. A heart-monitoring system, the system comprising:
at least two first-tier sensors capable of measuring at least two different aspects of the human body related to cardiac function and converting the at least two different aspects of the human body related to cardiac function into one or more signals;
at least one second-tier sensor that is also capable of being a first-tier sensor capable of measuring at least one aspect of the human body related to cardiac function and converting the at least one aspect of the human body related to cardiac function into one or more signals;
at least one signal processor capable of receiving and analyzing the one or more signals from the at least two first-tier sensors and the one or more signals from the at least one second-tier sensor and capable of transmitting or causing to be transmitted a first-tier trigger signal when coronary syndrome, cardiac ischemia or myocardial infarction has been detected in the human and a second-tier trigger signal when coronary syndrome, cardiac ischemia or myocardial infarction has been detected in the human;
at least one communication device capable of communicating any combination of the one or more signals from the at least two first-tier and one second-tier sensors, baseline cardiac function data regarding normal cardiac function of the human and threshold cardiac function data indicating coronary syndrome, cardiac ischemia or myocardial infarction in the human between the at least two first-tier sensors, the at least one second-tier sensor and the at least one signal processor;
at least one control element adapted to produce a first-tier trigger signal when at least one first-tier sensor exceeds its threshold signal level, produce a second-tier trigger signal when at least one second-tier sensor exceeds its threshold signal level after the first-tier trigger signal is produced, exclude the signal from the first-tier sensor that exceeded its threshold, producing the first-tier trigger signal and lower at least one threshold of the at least one first-tier sensor that is also a second-tier sensor when the first-tier trigger signal is produced.

2. The heart-monitoring system of claim 1, wherein one or more of the at least two first-tier sensors includes one or more electrocardiogram (ECG) or one or more intracardiac electrogram (EGM) sensors.

3. The heart-monitoring system of claim 1, wherein one of the at least two first-tier sensors is a patient activator with which the human may manually indicate the presence of a symptom suggesting a negative heart condition.

4. The heart-monitoring system of claim 1, wherein the at least two first-tier sensors and the at least one second tier sensor include is selected from a group of sensors that include: (1) an ECG sensor; (2) a heart sound sensor; (3) a vibration sensor; (4) a pressure sensor; (5) a change in pressure with time (dp/dt) sensor; (6) a magnetic sensor; (7) a hall sensor; (8) a biomarker level sensor; (9) an oxygen level sensor; (10) a carbon dioxide level sensor; (11) a glucose sensor; (12) a pH sensor; (13) an electrolyte sensor such as Na+, K+, or Ca+; (14) a temperature sensor; (15) a heart rate sensor; (16) a heart rate variability sensor; (17) a heart wall motion sensor; (18) an impedance sensor; (19) an optical or other type of electromagnetic radiation sensor; (20) a respiration sensor; (21) a 3-axis accelerometer; (22) a neurohormone sensor; (23) an external noise sensor; (24) a patient activator; and (25) optical color reflectometry sensor.

5. The ECG sensor of claim 4, containing electrodes to be placed on the surface of the skin, under the skin, or in or around the heart.

6. The heart sound sensor of claim 4, containing a microphone, accelerometer, or vibration sensor placed on the surface of the skin, under the skin, or in or around the heart.

7. The heart monitoring system of claim 1, wherein one or more tier-two sensors are not tier-one sensors, each additional tier-two sensor having a threshold being the signal level at or above which indicates coronary syndrome, cardiac ischemia or myocardial infarction in the human.

8. The heart monitoring system of claim 1, further comprising:
a second-tier initiation control element adapted to activate at least one of the at least one second-tier sensors when the first-tier trigger signal is produced.

9. The heart monitoring system of claim 1, further comprising:
a patient alert element adapted to produce an alert in response to the second-tier trigger signal.

10. The heart monitoring system of claim 1, further comprising:
an external system notification element adapted to send a message to an external system in response to the second-tier trigger signal.

11. The heart monitoring system of claim 1, further comprising a cardiac stimulus element.

12. The heart monitoring system of claim 11 adapted to produce a cardiac stimulus during the measurement period of the tier-two sensor in response to the tier-one trigger signal.

13. The heart monitoring system of claim 11 adapted to provide therapy to heart tissue of the human in response to the system detecting coronary syndrome, cardiac ischemia or myocardial infarction in the human.

14. A heart-monitoring apparatus adapted to detect coronary syndrome, cardiac ischemia or myocardial infarction in a human, the apparatus comprising:
one or more electrocardiogram (ECG) sensors adapted to sense bioelectrical activity, to produce one or more current ECG waveforms, and to produce an ECG trigger signal when at least a portion of one of the current ECG waveforms meets or exceeds a predetermined threshold level indicating coronary syndrome, cardiac ischemia or myocardial infarction in the human;
one or more heart sound sensors adapted to sense heart sounds, to produce a current heart sound waveforms, and to produce a heart sound trigger signal when at least a portion of one of the current heart sound waveforms meets or exceeds a predetermined threshold indicating coronary syndrome, cardiac ischemia or myocardial infarction in the human; and
a control element adapted to reduce the threshold for when at least a portion of the current ECG waveform indicates coronary syndrome, cardiac ischemia or myocardial infarction in the human when a heart sound trigger signal is produced and reduce the threshold for when at least a portion of the current heart sound waveform indicates coronary syndrome, cardiac ischemia or myocardial infarction in the human when an ECG trigger signal is produced.

15. The heart-monitoring apparatus of claim 14, wherein the one or more ECG sensors is adapted to produce the ECG trigger signal when a difference between an ST segment of the current ECG waveform and an ST segment of a baseline ECG waveform meets or exceeds a threshold value.

16. The heart-monitoring apparatus of claim 14, further comprising:
a patient alert element adapted to produce an alert in response or provide a heart-related therapy to the response-invoking signal.

17. A method for heart monitoring adapted to detect coronary syndrome, cardiac ischemia or myocardial infarction in the human, the method comprising the steps of:
sensing at least two first inputs related to cardiac function;
sensing at least one second input related to cardiac function when at least one first input indicates coronary syndrome, cardiac ischemia or myocardial infarction; and
producing a patient alert or providing a heart-related therapy when the at least one second input also indicates coronary syndrome, cardiac ischemia or myocardial infarction.

18. The method of claim 17, wherein sensing the at least two first inputs and at least one second input comprises the steps of:
sensing bioelectrical activity of a heart;
producing a current ECG waveform;
producing an ECG trigger signal when at least a portion of the current ECG waveform indicates the coronary syndrome, cardiac ischemia or myocardial infarction;
sensing heart sounds;
producing a current heart sound waveform; and
producing a heart sound trigger signal when at least a portion of the current heart sound waveform indicates the coronary syndrome, cardiac ischemia or myocardial infarction.

19. The method of claim 17, further comprising the step of:
determining that the at least one first input indicates coronary syndrome, cardiac ischemia or myocardial infarction in the human when a difference between the at least one first input and the baseline information exceeds a threshold;
adjusting at least one threshold used to determine whether the at least one second input indicates the coronary syndrome, cardiac ischemia or myocardial infarction;
sending a message to an internal or external notification system when the at least one second input also indicates coronary syndrome, cardiac ischemia or myocardial infarction.

20. The method of claim 19, further comprising the step of:
initiating cardiac stimulus or therapy when the at least one second input also indicates coronary syndrome, cardiac ischemia or myocardial infarction.
